(19) United States
(12) Patent Application Publication
(10) Pub. No.: US 2025/0144029 A1
(43) Pub. Date: May 8, 2025

(54) NOVEL COMPOSITIONS

(71) Applicant: INTRA-CELLULAR THERAPIES, INC., New York, NY (US)

(72) Inventor: Peng LI, New Milford, NJ (US)

(73) Assignee: INTRA-CELLULAR THERAPIES, INC., New York, NY (US)

(21) Appl. No.: 18/832,674

(22) PCT Filed: Jan. 27, 2023
(86) PCT No.: PCT/US2023/061469

§ 371 Date: Jul. 24, 2024

Related U.S. Application Data
(60) Provisional application No. 63/303,950, filed on Jan. 27, 2022.

(51) Int. Cl. A61K 9/14; A61K 31/519

(52) U.S. Cl. CPC A61K 9/146; A61K 9/145; A61K 31/519

(57) ABSTRACT

The present disclosure relates to amorphous solid dispersions comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one and an excipient, as well as related methods of making and using such dispersions.

### CROSS-REFERENCE TO RELATED APPLICATIONS

This application is an international application which claims priority to and the benefit of U.S. Provisional Application No. 63/303,950, filed on Jan. 27, 2022, the contents of which are incorporated herein in its entirety.

### FIELD OF THE DISCLOSURE

The present disclosure relates to amorphous solid dispersions containing a PDE1 inhibitor, e.g., (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one in free base or pharmaceutically acceptable salt form and an excipient, compositions comprising the same, as well as methods of making and using such amorphous solid dispersions.

### BACKGROUND OF THE DISCLOSURE

The compound (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one is disclosed in WO 2009/075784 (U.S. Pub. No. 2010/0273754). This compound has been found to be a potent and selective phosphodiesterase 1 (PDE 1) inhibitor useful for the treatment or prophylaxis of disorders characterized by low levels of cAMP and/or cGMP in cells expressing PDE1, and/or reduced dopamine D1 receptor signalling activity (e.g., neurodegenerative disorders such as Parkinson's disease; cognitive impairment of schizophrenia; cardiovascular disorders such as cardiac hypertrophy, heart failure and hypertension; cancers such as gliomas and leukaemia; and renal disorders such as kidney disease); and/or any disease or condition that may be ameliorated by the enhancement of progesterone signalling. This list of disorders is exemplary and not intended to be exhaustive.

Because many pharmaceutical compounds can exist in different physical forms (e.g., liquid or solid in different crystalline, amorphous, polymorphous, hydrate or solvate forms) which can affect the stability, solubility, bioavailability or pharmacokinetics (e.g., absorption, distribution, metabolism, excretion or the like) and/or bioequivalence of a drug, it is of critical importance in the pharmaceutical development to identify a pharmaceutical compound of optimal physical form (e.g., free base or salt in solid, liquid, crystalline, hydrate, solvate, amorphous or polymorphous forms).

The amorphous forms for many drugs exhibit superior dissolution characteristics and, in some cases, different bioavailability patterns compared to crystalline forms. Amorphous solid dispersions may be used for poorly soluble pharmaceutical compounds in development. These systems consist of an amorphous active pharmaceutical ingredient stabilized by a polymer or excipient to produce a system with improved physical and solution stability. Amorphous solid dispersions therefore may be used as a means of improving the solubility of an active pharmaceutical ingredient.

However, amorphous solid dispersions have been shown to be susceptible to changes during storage, and thus are not stable over time. For example, the amorphous solid dispersion of drug in excipient may separate into a drug-rich sections and/or convert over time to one or more crystalline polymorphs. The ideal drug formulation should be as physically stable as possible in a normal storage environment. Otherwise, such drug formulations may carry additional logistic requirement and/or burdensome restrictions on prescriptions and use by patients. A major problem with amorphous solid dispersions of drugs is that while the dispersions may show enhanced bioavailability of the low-solubility drug if administered shortly after preparation, bioavailability typically decreases over time in a typical storage environment. Such solid dispersions are often physically unstable in that the drug present in the dispersion reverts to the crystalline form upon storage-particularly at elevated temperature and humidity. Accordingly, the dispersion cannot be used to provide proper dosing of the drug because the bioavailability of the drug changes over time.

It is therefore desirable to procure compositions comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one in free base or pharmaceutically acceptable salt form and an excipient which have enhanced characteristics, such as solubility and/or bioavailability. It is also desirable for such compositions to be storage stable for extended periods of time.

### SUMMARY OF THE DISCLOSURE

Provided herein are compositions (e.g., amorphous solid dispersions) comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one which have enhanced characteristics, such as solubility and/or bioavailability. Further provided herein are compositions (i.e., amorphous solid dispersions) which are storage stable for extended periods of time.

In a first aspect, the present disclosure provides an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free base or pharmaceutically acceptable salt form and an excipient. In certain embodiments, the excipient is one or more of a cellulose; polyethylene glycol (e.g., polyethylene glycol having a molecular weight of 500-50000 Daltons, e.g., PEG 1000 or PEG 10000); monostearine; polyvinyl pyrrolidone; PEG/PPG block co-polymers (e.g., a poloxamer (e.g., poloxamer 407, Pluronic F127)); ascorbic acid (i.e., L-ascorbic acid); butylated hydroxyanisole; sodium dodecyl sulfate; a cyclodextrin (e.g., beta cyclodextrin, (2-hydroxypropyl) beta-cyclodextrin); or combinations thereof. In various embodiments, the composition is stable for an extended period of time (e.g., for a period of about one day, about three days, or about 7 days, e.g., under accelerated aging conditions, e.g., 40° C. and 75% relative humidity).

In a second aspect, the present disclosure provides a method of making an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) and an excipient, the method comprising the steps of:
a) dissolving Compound 1 in a first solvent;
b) dissolving the excipient in a second solvent;
c) combining the solutions from steps a) and b); and
d) removing the solvents from the mixture.
   In certain embodiments, the step of removing the solvents comprises lyophilization and/or evaporation. That is, the step of removing the solvents may include freezing the mixture at a temperature at or below 0° C., e.g., at or below −10° C., e.g., at or below −20° C., followed by evaporation of the solvent at reduced pressure (e.g., 0 bar), e.g., at room temperature.

In a third aspect, the present disclosure provides an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free base or pharmaceutically acceptable salt form and an excipient, which is obtained or obtainable by the method comprising the steps of:
a) dissolving Compound 1 in a first solvent;
b) dissolving the excipient in a second solvent;
c) combining the solutions from steps a) and b); and
d) removing the solvents from the mixture.

In a fourth aspect, the present disclosure is directed to a method for the prophylaxis or treatment of a patient, e.g., a human, suffering from a disorder selected from one or more of a neurodegenerative disease; a mental disorder; a circulatory or cardiovascular disorder; a respiratory or inflammatory disorder; a diseases which may be alleviated by the enhancement of progesterone-signalling such as female sexual dysfunction; glaucoma or elevated intraocular pressure; traumatic brain injuries; a cancers or tumor; a renal disorder; any disease or condition characterized by low levels of cAMP and/or cGMP (or inhibition of cAMP and/or cGMP signaling pathways) in cells expressing PDE1; or any disease or condition characterized by reduced dopamine D1 receptor signaling activity, wherein the method comprises administering to a patient in need thereof a therapeutically effective amount of an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) and an excipient. In certain embodiments, Compound 1 is administered in an amount of about 0.5 mg to about 300 mg, wherein the amount is calculated as the free base equivalent.

Additional features and advantages of various embodiments will be set forth in part in the description that follows, and in part will be apparent from the description and examples. The objectives and other advantages of various embodiments will be realized and attained by means of the elements and combinations particularly pointed out in the description and appended claims.

### DETAILED DESCRIPTION

As used herein, the term “amorphous” means a solid without long-range crystalline order. The term “amorphous” form refers to solids of disordered arrangements of molecules and do not possess a distinguishable crystal lattice. The amorphous form of (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in accordance with the present disclosure preferably contains less than about 10 wt. %, preferably less than 5 wt. %, more preferably less than about 2 wt. %, still preferably less than about 1 wt. %, still preferably less than about 0.1%, most preferably less than about 0.01 wt. %, and more preferably is essentially free of crystalline forms of Compound 1. “Essentially free of crystalline forms of Compound 1” means that no crystalline polymorph forms of Compound 1 can be detected within the limits of an X-ray powder diffractometer.

As used herein, the term “dispersion” refers to a disperse system in which one substance, the dispersed phase, is distributed, in discrete units, throughout a second substance (i.e., a continuous phase or vehicle). The size of the dispersed phase can vary (e.g., colloidal particles ranging from nanometer scale up to microns scale in size). As relates to the amorphous solid dispersions of the present disclosure, the dispersed and continuous phases are both solids. In pharmaceutical applications, a solid dispersion can include a crystalline drug (dispersed phase) in an amorphous excipient (continuous phase), or alternatively, an amorphous drug (dispersed phase) in an amorphous excipient (continuous phase). It is envisioned that in some embodiments the amorphous solid dispersion of the present disclosure includes the excipient constituting the dispersed phase, and the drug constitutes the continuous phase. In other embodiments, the dispersion includes amorphous Compound 1 or substantially amorphous Compound 1.

The term “amorphous solid dispersion” generally refers to a solid dispersion of two or more components, usually a drug and polymer, but possibly containing other components such as surfactants or other pharmaceutical excipients, where Compound 1 is amorphous or substantially amorphous (e.g., substantially free of crystalline Compound 1), and the physical stability and/or dissolution and/or solubility of the amorphous drug is enhanced by the other components.

As used herein, the term “crystal” or “crystals” or “crystalline” or “crystallinic” refers to any solid that has a short- or long-range order of the molecules, atoms or ions in a fixed lattice arrangement.

The amorphous solid dispersions of the present disclosure may have a Compound 1 to excipient ratio of between about 5:1 and 1:5. For example, in various embodiments, the ratio of Compound 1 to excipient may be between 1:2 and 2:1, e.g., 1:1, 1:2, or 2:1.

Unless further modified, the term “Compound 1” refers to (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one in free base form, having the following structure:

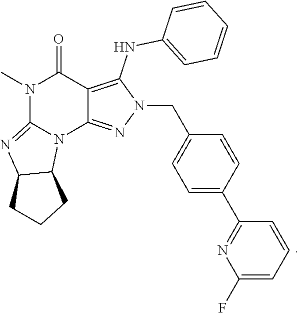 [SMILES: CN1C(=O)c2c(nn(Cc3ccc(-c4cccc(F)n4)cc3)c2Nc2ccccc2)N2C1=N[C@@H]1CCC[C@@H]12]

The crystallinity or the morphology of the amorphous forms of the present disclosure may be determined by a number of methods, including, but not limited to single crystal X-ray diffraction, X-ray powder diffraction, polarizing optical microscopy, thermal microscopy, differential scanning calorimetry (DSC), thermogravimetric analysis (TGA), infrared adsorption spectroscopy and Raman spectroscopy.

The term “about” in front of a numerical value refers to approximations that may vary depending upon the desired properties sought to be obtained by the present application. Each numerical value disclosed herein may at least be construed in light of ordinary rounding techniques. When referencing temperature, the term about refers to the temperature value itself±10° C., preferably ±5° C., preferably ±3° C. of the reference temperature.

The amorphous solid dispersions of the disclosure comprise selective PDE1 inhibitors. Therefore, the amorphous solid dispersions of the disclosure are useful for the treatment of PDE1 related disorders as set forth in e.g., WO 2014/151409, WO 2018/049417, WO 2019/227004, WO 2019/152697, WO 2009/075784, WO 2010/132127, WO 2006/133261 and WO 2011/153129, the contents of each of which are incorporated by reference in their entireties.

The term “patient” includes human and non-human. In one embodiment, the patient is a human. In another embodiment, the patient is a non-human.

In a first aspect, the present disclosure provides an amorphous solid dispersion [Dispersion 1] comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) and an excipient. For example, the present disclosure provides for the following embodiments of Dispersion 1:
1.1 Dispersion 1, wherein Compound 1 is in free base or pharmaceutically acceptable salt form.
1.2 Dispersion 1 or 1.1, wherein Compound 1 is in free base form.
1.3 Any of the preceding Dispersions, wherein Compound 1 is in salt form.
1.4 Any of the preceding Dispersions, wherein Compound 1 is in phosphate salt form.
1.5 Any of the preceding Dispersions, wherein Compound 1 is in mono-phosphate salt form.
1.6 Any of the preceding Dispersions, wherein the excipient comprises a cellulose; polyethylene glycol (e.g., polyethylene glycol having a molecular weight of 500-50000 Daltons, e.g., PEG 1000 or PEG 10000); monostearine; polyvinyl pyrrolidone; PEG/PPG block co-polymers (e.g., a poloxamer (e.g., poloxamer 407, Pluronic F127)); ascorbic acid (i.e., L-ascorbic acid); butylated hydroxyanisole; sodium dodecyl sulfate; a cyclodextrin (e.g., beta cyclodextrin, 2-Hydroxy propyl)-β-cyclodextrin); or combinations thereof.
1.7 Any of the preceding Dispersions, wherein the excipient comprises cellulose; hydroxypropyl cellulose; methyl cellulose; hydroxy propyl methyl cellulose; sodium dodecyl sulfate; ascorbic acid (e.g., L-ascorbic acid); beta-cyclodextrin; (2-hydroxypropyl) beta-cyclodextrin; cellulose acetate; cellulose acetate phthalate; polyethylene glycol (e.g., polyethylene glycol having a molecular weight of 500-50000 Daltons, e.g., PEG 1000 or PEG 10000); PEG/PPG block co-polymers (e.g., a poloxamer (e.g., poloxamer 407, e.g., Pluronic F127)); butylated hydroxyanisole; monostearine; or combinations thereof.
1.8 Any of the preceding Dispersions, wherein the excipient consists of a member of the group selected from a cellulose; polyethylene glycol (e.g., polyethylene glycol having a molecular weight of 500-50000 Daltons, e.g., PEG 1000 or PEG 10000); monostearine; polyvinyl pyrrolidone; PEG/PPG block co-polymers (e.g., a poloxamer (e.g., poloxamer 407, pluronic F127)); ascorbic acid (i.e., L-ascorbic acid); butylated hydroxyanisole; sodium dodecyl sulfate; a cyclodextrin (e.g., beta cyclodextrin, (2-Hydroxy propyl)-β-cyclodextrin); and combinations thereof.
1.9 Any of the preceding Dispersions, wherein the excipient consists of a member of the group selected from cellulose; hydroxypropyl cellulose; methyl cellulose; hydroxy propyl methyl cellulose; sodium dodecyl sulfate; ascorbic acid (e.g., L-ascorbic acid); beta-cyclodextrin; (2-hydroxypropyl) beta-cyclodextrin; cellulose acetate; cellulose acetate phthalate; polyethylene glycol (e.g., polyethylene glycol having a molecular weight of 500-50000 Daltons, e.g., PEG 1000 or PEG 10000); PEG/PPG block co-polymers (e.g., a poloxamer (e.g., poloxamer 407, Pluronic F127)); butylated hydroxyanisole; monostearine; and combinations thereof.
1.10 Any of the preceding Dispersions, wherein the excipient comprises one or more of a cellulose; a cyclodextrin; polyvinyl pyrrolidone; ascorbic acid (e.g., L-ascorbic acid), or combinations thereof.
1.11 Any of the preceding Dispersions, wherein the excipient comprises one or more of cellulose, methyl cellulose; hydroxy propyl methyl cellulose; (2-hydroxypropyl) beta-cyclodextrin; cellulose acetate; cellulose acetate phthalate; polyvinyl pyrrolidone (e.g., K85-K95); ascorbic acid (e.g., L-ascorbic acid); or combinations thereof.
1.12 Any of the preceding Dispersions, wherein the excipient is selected from the group consisting of cellulose; methyl cellulose; hydroxy propyl methyl cellulose; (2-hydroxypropyl) beta-cyclodextrin; cellulose acetate; cellulose acetate phthalate; polyvinyl pyrrolidone (e.g., K85-K95); ascorbic acid (e.g., L-ascorbic acid); and combinations thereof.
1.13 Any of the preceding Dispersions, wherein Compound 1 and the excipient are present in a molar ratio between 1:5 to 5:1.
1.14 Any of the preceding Dispersions, wherein Compound 1 and the excipient are present in a molar ratio between 1:2 to 2:1.
1.15 Any of the preceding Dispersions, wherein Compound 1 and the excipient are present in a molar ratio of 1:1.
1.16 Any of the preceding Dispersions, wherein Compound 1 and the excipient are present in a molar ratio of 1:2.
1.17 Any of the preceding Dispersions, wherein Compound 1 and the excipient are present in a molar ratio of 2:1.
1.18 Any of the preceding Dispersions, wherein the amorphous solid dispersion is in the form of a powder.
1.19 Any of the preceding Dispersions, wherein the amorphous solid dispersion is present as the active ingredient in a tablet, e.g., oral disintegrating tablet (ODT), extended-release tablet, enteric tablet, capsule, e.g., extended-release capsule, enteric capsule, multiparticulate, or injectable suspension.
1.20 Any of the preceding Dispersions, wherein the amorphous solid dispersion is in dosage form, which contains Compound 1 in an amount of about 0.5 mg to about 300 mg, wherein the amount is calculated as the free base equivalent.
1.21 Any of the preceding Dispersions, wherein the amorphous solid dispersion is in dosage form, which contains Compound 1 in an amount of about 1 mg to about 100 mg, wherein the amount is calculated as the free base equivalent.
1.22 Any of the preceding Dispersions, wherein the amorphous solid dispersion is in dosage form, which contains Compound 1 in an amount of about 10 mg to about 90 mg, wherein the amount is calculated as the free base equivalent.
1.23 Any of the preceding Dispersions, wherein Compound 1 is administered in an amount of about 30 mg to about 90 mg, wherein the amount is calculated as the free base equivalent.
1.24 Any of the preceding Dispersions, wherein the amorphous solid dispersion is in dosage form, which contains Compound 1 in an amount of about 10 mg, about 30 mg, or about 90 mg, wherein the amount is calculated as the free base equivalent.
1.25 Any of the preceding Dispersions, wherein the amorphous solid dispersion is in dosage form, which contains Compound 1 in an amount of about 30 mg, wherein the amount is calculated as the free base equivalent.
1.26 Any of the preceding Dispersions, wherein the amorphous solid dispersion is stable for a period of about one day, about three days, or about 7 days.
1.27 Any of the preceding Dispersions, wherein the amorphous solid dispersion is stable for a period of about one day, about three days, or about 7 days under accelerated aging conditions (e.g., 40° C. and 75% relative humidity).
1.28 Any of the preceding Dispersions, wherein the amorphous solid dispersion is stable for a period of about 7 days under accelerated aging conditions (e.g., 40° C. and 75% relative humidity).
1.29 Any of the preceding Dispersions, wherein the amorphous solid dispersion is formed through the use of lyophilization.
1.30 Any of the preceding Dispersions, wherein the amorphous solid dispersion is formed through the use of evaporation.
1.31 Any of the preceding Dispersions, wherein Compound 1 is in free base form and the excipient is selected from cellulose; hydroxypropyl cellulose; methyl cellulose; hydroxy propyl methyl cellulose; (2-hydroxypropyl) beta-cyclodextrin; cellulose acetate phthalate; polyvinyl pyrrolidone; and combinations thereof, wherein the molar ratio of Compound 1 to the excipient is 1:1, 1:2 or 2:1, and optionally wherein the amorphous solid dispersion is formed through the use of lyophilization.
1.32 Any of the preceding Dispersions, wherein Compound 1 is in free base form and the excipient is selected from cellulose; methyl cellulose; hydroxy propyl methyl cellulose; (2-hydroxypropyl) beta-cyclodextrin; cellulose acetate; L-ascorbic acid; and combinations thereof, wherein the molar ratio of Compound 1 to the excipient is 1:1, 1:2 or 2:1, and optionally wherein the amorphous solid dispersion is formed through the use of evaporation.
1.33 Any of the preceding Dispersions, wherein Compound 1 is in the form of a phosphate salt (e.g., a mono-phosphate salt) and the excipient is selected from cellulose; methyl cellulose; hydroxy propyl methyl cellulose; cellulose acetate; and combinations thereof, wherein the molar ratio of Compound 1 to the excipient is 1:1, 1:2 or 2:1, and optionally wherein the amorphous solid dispersion is formed through the use of lyophilization.
1.34 Any of the preceding Dispersions, wherein Compound 1 is in the form of a phosphate salt (e.g., a mono-phosphate salt) and the excipient is selected from cellulose; hydroxypropyl cellulose; methyl cellulose; hydroxy propyl methyl cellulose; (2-hydroxypropyl) beta-cyclodextrin; cellulose acetate; cellulose acetate phthalate; polyvinyl pyrrolidone; and combinations thereof, wherein the molar ratio of Compound 1 to the excipient is 1:1, 1:2 or 2:1, and optionally wherein the amorphous solid dispersion is formed through the use of evaporation.
1.35 Any of the preceding Dispersions, wherein Compound 1 is in substantially amorphous solid form.
1.36 Any of the preceding Dispersions, wherein Compound 1 is amorphous and contains less than 10 wt. % crystalline forms of Compound 1.
1.37 Any of the preceding Dispersions, wherein Compound 1 is amorphous and contains less than 5 wt. % crystalline forms of Compound 1.
1.38 Any of the preceding Dispersions, wherein Compound 1 is amorphous and contains less than 1 wt. % crystalline forms of Compound 1.
1.39 Any of the preceding Dispersions, wherein Compound 1 is amorphous and contains less than 0.1 wt. % crystalline forms of Compound 1.
1.40 Any of the preceding Dispersions, wherein Compound 1 is amorphous and contains less than 0.01 wt. % crystalline forms of Compound 1.
1.41 Any of the preceding Dispersions, wherein Compound 1 is amorphous and is essentially free of crystalline forms of Compound 1.
1.42 Any of the preceding Dispersions, wherein the excipient is amorphous.

In a second aspect, the present disclosure provides a method [Method 1] of making an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) and an excipient (e.g., Dispersion 1, et seq.), the method comprising the steps of:
a) dissolving Compound 1 in a first solvent;
b) dissolving the excipient in a second solvent;
c) combining the solutions from steps a) and b); and
d) removing the solvents from the mixture.

For example, the present disclosure provides for the following embodiments of Method 1:
1.1 Method 1, wherein the amorphous solid dispersion is according to any of Dispersion 1, et seq.
1.2 Method 1 or 1.1, wherein the first solvent comprises one or more of water, acetone, dichloromethane, an alcohol (e.g., methanol or ethanol), dioxane, tetrahydrofuran, acetonitrile, and combinations thereof.
1.3 Any of the preceding Methods, wherein the first solvent is acetone; dichloromethane; dioxane; dioxane and methanol (e.g., in a 1:1 ratio); acetone and ethanol (e.g., in a 3:1 ratio); dioxane and water (e.g., in a 9:1 ratio); tetrahydrofuran and water (e.g., in a 9:1 ratio); methanol and water (e.g., in a ratio of 9:1); or acetonitrile and water (e.g., in a ratio of 9:1).
1.4 Any of the preceding methods, wherein the first solvent is acetone; dichloromethane; or acetone and ethanol (e.g., in a 3:1 ratio).
1.5 Any of the preceding methods, wherein the first solvent is tetrahydrofuran and water (e.g., in a 9:1 ratio); methanol and water (e.g., in a ratio of 9:1); or acetonitrile and water (e.g., in a ratio of 9:1).
1.6 Any of the preceding methods, wherein the second solvent is water and/or methanol.
1.7 Any of the preceding methods, wherein removing the solvents from the mixture comprises lyophilization and/or evaporation.
1.8 The preceding method, wherein the removal step comprises lyophilization and evaporation.
1.9 The preceding method, wherein the removal step comprises freezing the mixture at a temperature at or below 0° C., e.g., at or below −10° C., e.g., at or below −20° C., followed by evaporation of the solvent at reduced pressure (e.g., 0 bar), e.g., at room temperature.
1.10 Any of Method 1 or 1.1-1.7, wherein the removal step comprises evaporation of the solvent at reduced pressure (e.g., 0 bar), e.g., at room temperature.

In a third aspect, the present disclosure provides for an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) and an excipient according to Dispersion 1, et seq., which is obtained or obtainable by the methods according to Method 1, et seq.

In a fourth aspect, the present disclosure further provides a method [Method 2] for the prophylaxis or treatment of a patient, e.g., a human, suffering from a disorder selected from the following:
A. Neurodegenerative diseases, including Parkinson's disease, restless leg, tremors, dyskinesias, Huntington's disease, Alzheimer's disease, and drug-induced movement disorders;
B. Mental disorders, including depression, attention deficit disorder, attention deficit hyperactivity disorder, bipolar illness, anxiety, sleep disorders, e.g., narcolepsy, cognitive impairment, e.g., cognitive impairment of schizophrenia, dementia, Tourette's syndrome, autism, fragile X syndrome, psychostimulant withdrawal, and drug addiction;
C. Circulatory and cardiovascular disorders, including cerebrovascular disease, stroke, congestive heart disease, hypertension, pulmonary hypertension, e.g., pulmonary arterial hypertension, and sexual dysfunction, including cardiovascular diseases and related disorders as described in International Application No. PCT/US2014/16741, the contents of which are incorporated herein by reference;
D. Respiratory and inflammatory disorders, including asthma, chronic obstructive pulmonary disease, and allergic rhinitis, as well as autoimmune and inflammatory diseases;
E. Diseases that may be alleviated by the enhancement of progesterone-signaling such as female sexual dysfunction;
F. A disease or disorder such as psychosis, glaucoma, or elevated intraocular pressure;
G. Traumatic brain injury;
H. Cancers or tumors, e.g., brain tumors, a glioma (e.g., ependymoma, astrocytoma, oligodendrogliomas, brain stem glioma, optic nerve glioma, or mixed gliomas, e.g., oligoastrocytomas), an astrocytoma (e.g., glioblastoma multiforme), osteosarcoma, melanoma, leukemia, neuroblastoma or leukemia;
I. Renal disorders, e.g., kidney fibrosis, chronic kidney disease, renal failure, glomerulosclerosis and nephritis;
J. Any disease or condition characterized by low levels of cAMP and/or cGMP (or inhibition of cAMP and/or cGMP signaling pathways) in cells expressing PDE1; and/or
K. Any disease or condition characterized by reduced dopamine D1 receptor signaling activity,
   wherein the method comprises administering to a patient in need thereof a therapeutically effective amount of an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) and an excipient according, e.g., Dispersion 1 et seq., of the present disclosure. For example, the present disclosure further provides the following embodiments.
2.1 Any of the preceding Methods, wherein the amorphous solid dispersion is in the form of a powder.
2.2 Any of the preceding Methods, wherein the amorphous solid dispersion is present as the active ingredient in a tablet, e.g., oral disintegrating tablet (ODT), extended-release tablet, enteric tablet, capsule, e.g., extended release capsule, enteric capsule, multiparticulate, or injectable suspension.
2.3 Any of the preceding Methods, wherein Compound 1 is administered in an amount of about 0.5 mg to about 300 mg, wherein the amount is calculated as the free base equivalent.
2.4 Any of the preceding Methods, wherein Compound 1 is administered in an amount of about 1 mg to about 100 mg, wherein the amount is calculated as the free base equivalent.
2.5 Any of the preceding Methods, wherein Compound 1 is administered in an amount of about 10 mg to about 90 mg, wherein the amount is calculated as the free base equivalent.
2.6 Any of the preceding Methods, wherein Compound 1 is administered in an amount of about 30 mg to about 90 mg, wherein the amount is calculated as the free base equivalent.
2.7 Any of the preceding Methods, wherein Compound 1 is administered in an amount of about 10 mg, about 30 mg, or about 90 mg, wherein the amount is calculated as the free base equivalent.
2.8 Any of the preceding Methods, wherein Compound 1 is administered in an amount of about 30 mg, wherein the amount is calculated as the free base equivalent.

A pharmaceutical composition comprising Dispersion 1, et seq., for use as a medicament, e.g., for use in the manufacture of a medicament for the treatment or prophylaxis of a disease as described in Method 2, et seq.

The disclosure further provides Dispersion 1, et seq., for use in any of Methods 2, et seq.

The disclosure further provides the use Dispersion 1, et seq., in the manufacture of a medicament for use in any of Methods 2, et seq.

Without being bound by theory, it is believed that the amorphous solid dispersions of the present disclosure are substantially homogeneous such that the amorphous PDE1 inhibitor is dispersed as homogeneously as possible throughout the excipient. As used herein, “substantially homogeneous” means that the presence of pure amorphous domains within the solid dispersion are minor or non-existent. For example, the proportion of amorphous domains of the PDE1 inhibitor account for less than 10% of the amorphous solid dispersion, preferably less than 5% of the amorphous solid dispersion, more preferably less than 1% of the amorphous solid dispersion, more preferably such domains are not present. While the dispersion may have some drug-rich domains, it is preferred that the dispersion itself have a single glass transition temperature which demonstrates that the dispersion is substantially homogenous. By contrast a physical mixture of pure amorphous drug particles and pure amorphous excipient particles will generally display two distinct glass transition temperatures.

The particle size and the temperature drying range may be modified to prepare an optimal solid dispersion. In general, small particle size leads to improved solvent removal. In general, particle size may vary from nanometer scale up to microns scale in size.

The present disclosure further provides for pharmaceutically acceptable compositions, wherein such compositions comprise the amorphous solid dispersions as described, and optionally comprise a pharmaceutically acceptable carrier, adjuvant or vehicle. In certain embodiments, these compositions optionally further comprise one or more additional therapeutic agents.

It will also be appreciated that Compound 1 can exist in free form for treatment, or where appropriate, as a pharmaceutically acceptable derivative or a prodrug thereof. According to the present disclosure, a pharmaceutically acceptable derivative or a prodrug includes, but is not limited to, pharmaceutically acceptable salts, esters, salts of such esters, or any other adduct or derivative which upon administration to a patient in need thereof is capable of providing, directly or indirectly, a compound as otherwise described herein, or a metabolite or residue thereof.

As used herein, the term “pharmaceutically acceptable salt” refers to any salt which are suitable for use in contact with the tissues of humans and/or animals without undue toxicity, irritation, allergic response and the like, and are commensurate with a reasonable benefit/risk ratio. A “pharmaceutically acceptable salt” means any non-toxic salt or salt of an ester of Compound 1 that, upon administration to a recipient, is capable of providing, either directly or indirectly, Compound 1 or an active metabolite or residue thereof.

Pharmaceutically acceptable salts of Compound 1 as described herein include those derived from suitable inorganic and organic acids and bases. Non-limiting examples of pharmaceutically acceptable salts are fumarate, hydrochloric, (1-hydrox-2)-naphthoate, benzosulfonate, phosphate, mesylate, tartrate, sulphate and hydrobromate salts.

The pharmaceutically acceptable compositions of the present disclosure may additionally comprise a pharmaceutically acceptable carrier, adjuvant, or vehicle, which, as used herein, includes any and all solvents, diluents, or other liquid vehicle, dispersion or suspension aids, surface active agents (i.e., surfactants), isotonic agents, thickening or emulsifying agents, preservatives, solid binders, lubricants and the like, as suited to the particular dosage form desired. The skilled artisan will appreciate that any conventional carrier medium or additives which are compatible with the amorphous solid dispersions of the present disclosure may be used in such compositions. Some examples of materials which can serve as pharmaceutically acceptable carriers include, but are not limited to, ion exchangers, alumina, aluminium stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition.

Non-limiting examples of suitable surfactants include fatty acid and alkyl sulfonates; commercial surfactants such as sorbitan fatty acid esters; polyoxyethylene sorbitan fatty acid esters; sodium lauryl sulfate (SLS); sodium dodecylbenzene sulfonate (SDBS) dioctyl sodium sulfosuccinate; dioxycholic acid sodium salt (DOSS); sorbitan monostearate; sorbitan tristearate; hexadecyltrimethyl ammonium bromide (HTAB); sodium N-lauroylsarcosine; sodium oleate; sodium myristate; sodium stearate; sodium palmitate; gelucire 44/14; ethylenediamine tetraacetic acid (EDTA); vitamin E d-alpha tocopheryl polyethylene glycol 1000 succinate (TPGS); lecithin; glutamic acid monosodium monohydrate; labrasol; PEG 8 caprylic/capric glycerides; transcutol; diethylene glycol monoethyl ether; solutol HS-15; polyethylene glycol/hydroxystearate; taurocholic acid, pluronic F68; pluronic F108; Pluronic F127 (or any other polyoxyethylene-polyoxypropylene co-polymers); or saturated polyglycolized glycerides (Gelucirs®)).

Addition of pH modifiers such as acids, bases, or buffers may also be beneficial, retarding the dissolution of the dispersion or, alternatively, enhancing the rate of dissolution of the dispersion. Addition of conventional matrix materials, surfactants, fillers, disintegrants, or binders may be added as part of the dispersion itself, added by granulation via wet, mechanical, or other means.

Examples of other matrix materials, fillers, or diluents include lactose, mannitol, xylitol, microcrystalline cellulose, calcium diphosphate, starch, polyoxamers such as polyethylene oxide, and hydroxypropyl methyl cellulose. Examples of drug complexing agents or solubilizers include the polyethylene glycols, caffeine, xanthene, gentisic acid and cylodextrins. Examples of disintegrants include sodium starch glycolate, sodium alginate, carboxy methyl cellulose sodium, methyl cellulose, and croscarmellose sodium. Examples of binders include methyl cellulose, microcrystalline cellulose, starch, and gums such as guar gum, and tragacanth. Examples of lubricants include magnesium stearate and calcium stearate. Exemplary pH modifiers include acids such as citric acid, acetic add, ascorbic acid, lactic acid, tartaric acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like; and buffers generally comprising mixtures of acids and the salts of said acids.

In some embodiments, the compositions may be coated with an enteric polymer to prevent or retard dissolution until the dosage form leaves the stomach. Exemplary enteric coating materials include HPMCAS, HPMCP, cellulose acetate phthalate, cellulose acetate trimellitate, carboxylic acid-functionalized polymethacrylates, and carboxylic acid-functionalized polyacrylate.

In addition to the above additives or excipients, use of any conventional materials and procedures for formulation and preparation of various dosage forms using the compositions of this disclosure known by those skilled in the art are potentially useful.

The pharmaceutically acceptable compositions of this disclosure can be administered to humans and other animals orally, rectally, parenterally, intracisternally, intravaginally, intraperitoneally, topically (as by powders, ointments, or drops), bucally, as an oral or nasal spray, or the like.

In some embodiments, compositions of the present disclosure may be used in a wide variety of forms for administration of drugs orally. Exemplary dosage forms are powders or granules that may be taken orally either dry or reconstituted by addition of water to form a paste, slurry, suspension or solution; tablets, e.g., oral disintegrating tablets (ODT), extended-release tablets, enteric tablets, capsules, e.g., extended-release capsules, enteric capsules, multiparticulates or pills. Various additives may be mixed, ground, or granulated with the compositions of this disclosure to form a material suitable for the above dosage forms. Potentially beneficial additives fall generally into the following classes: other matrix materials or diluents, surfactants, drug complexing agents or solubilizers, fillers, disintegrants, binders, lubricants, and pH modifiers (e.g., acids, bases, or buffers).

Liquid dosage forms for oral administration include, but are not limited to, pharmaceutically acceptable emulsions, micro emulsions, solutions, suspensions, syrups and elixirs. In addition to the active compounds, the liquid dosage forms may contain inert diluents commonly used in the art such as, for example, water or other solvents, solubilizing agents and emulsifiers such as water, acetone, dichloromethane, an alcohol (e.g., methanol or ethanol), dioxane, tetrahydrofuran, acetonitrile, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and combinations thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium.

Other features and embodiments of the disclosure will become apparent from the following examples which are given for illustration of the disclosure rather than for limiting its intended scope.

### EXAMPLES

### Example 1: Free Base Containing Amorphous Solid Dispersions Created Using Lyophilization

5 mg of the Compound 1 Free Base is dissolved in acetone (500 μl) or dichloromethane (500 μl) and the excipients are dissolved in either water (500 μl) or methanol (500 μl), depending on their solubility. The Free Base solution and excipient solution are added to each other. Some drops of water are added for the experiments that did not contain water. The samples are subsequently stored in a freezer (−20° C.) for 16 hours and thereafter stored in vacuum (0 mbar, RT) for quick evaporation of the solvents. The experiments are performed using molar ratios of Free Base to excipient of 1:1, 1:2 and 2:1. Visual appearance is recorded and XRPD is measured for observed solids. The experiments marked with an “N/A” were not carried out due to immiscibility between dichloromethane and water.

TABLE 1
Lyophilization experiments with Free Base:excipient ratio of 1:1

| | | Molar Ratio | | |
|---|---|---|---|---|
| | Excipient | (Free | Experimental Results | |
| Excipient | Solvent | Base:Excipient) | Acetone | Dichloromethane |
| Cellulose | Methanol | 1:1 | Amorphous | Excipient only |
| Hydroxypropyl | Methanol | 1:1 | Glass/oily | Amorphous |
| Cellulose | | | substance | |
| Methyl | Water | 1:1 | Amorphous | N/A |
| Cellulose | | | | |
| Hydroxy Propyl | Water | 1:1 | Glass/oily | N/A |
| Methyl | | | substance | |
| Cellulose | | | | |
| Sodium dodecyl | Methanol | 1:1 | Glass/oily | Glass/oily |
| Sulfate | | | substance | substance |
| L-Ascorbic Acid | Methanol | 1:1 | Glass/oily | Glass/oily |
| | | | substance | substance |
| Beta- | Water | 1:1 | Excipient only | N/A |
| Cyclodextrin | | | | |
| (2- | Methanol | 1:1 | Amorphous | Amorphous |
| hydroxypropyl) | | | | |
| Beta- | | | | |
| Cyclodextrin | | | | |
| Cellulose acetate | Methanol | 1:1 | Glass/oily | Glass/oily |
| | | | substance | substance |
| Cellulose acetate | Methanol | 1:1 | Amorphous | Glass/oily |
| phthalate | | | | substance |
| PEG 1000 | Methanol | 1:1 | Glass/oily | Glass/oily |
| | | | substance | substance |
| PEG 10000 | Water | 1:1 | Excipient only | N/A |
| Polyvinyl | Methanol | 1:1 | Amorphous | Glass/oily |
| Pyrrolidone | | | | substance |
| (K85-K95) | | | | |
| Poloxamer 407 | Methanol | 1:1 | Glass/oily | Glass/oily |
| (Pluronic F127) | | | substance | substance |
| Butylated | Methanol | 1:1 | Glass/oily | Glass/oily |
| hydroxyanisole | | | substance | substance |
| Monostearine | Methanol | 1:1 | Excipient only | Poor crystallinity |

TABLE 2
Lyophilization experiments with Free Base:excipient ratio of 1:2

| | | Molar Ratio | | |
|---|---|---|---|---|
| | Excipient | (Free | Experimental Results | |
| Excipient | Solvent | Base:Excipient) | Acetone | Dichloromethane |
| Cellulose | Methanol | 1:2 | Amorphous | Excipient only |
| Hydroxypropyl | Methanol | 1:2 | Glass/oily | Glass/oily |
| Cellulose | | | substance | substance |
| Methyl | Water | 1:2 | Amorphous | N/A |
| Cellulose | | | | |
| Hydroxy Propyl | Water | 1:2 | Amorphous | N/A |
| Methyl | | | | |
| Cellulose | | | | |
| Sodium dodecyl | Methanol | 1:2 | Excipient only | Excipient only |
| Sulfate | | | | |
| L-Ascorbic Acid | Methanol | 1:2 | Glass/oily | Glass/oily |
| | | | substance | substance |
| Beta- | Water | 1:2 | Crystal form | N/A |
| Cyclodextrin | | | | |
| (2- | Methanol | 1:2 | Amorphous | Amorphous |
| hydroxypropyl) | | | | |
| Beta- | | | | |
| Cyclodextrin | | | | |
| Cellulose acetate | Methanol | 1:2 | Glass/oily | Glass/oily |
| | | | substance | substance |
| Cellulose acetate | Methanol | 1:2 | Glass/oily | Glass/oily |
| phthalate | | | substance | substance |
| PEG 1000 | Methanol | 1:2 | Excipient only | Excipient only |
| PEG 10000 | Water | 1:2 | Excipient only | N/A |
| Polyvinyl | Methanol | 1:2 | Glass/oily | Glass/oily |
| Pyrrolidone | | | substance | substance |
| (K85-K95) | | | | |
| Poloxamer 407 | Methanol | 1:2 | Excipient only | Glass/oily |
| (Pluronic F127) | | | | substance |
| Butylated | Methanol | 1:2 | Glass/oily | Glass/oily |
| hydroxyanisole | | | substance | substance |
| Monostearine | Methanol | 1:2 | Excipient only | Excipient only |

TABLE 3
Lyophilization experiments with Free Base:excipient ratio of 2:1

| | | Molar Ratio | | |
|---|---|---|---|---|
| | Excipient | (Free | Experimental Results | |
| Excipient | Solvent | Base:Excipient) | Acetone | Dichloromethane |
| Cellulose | Methanol | 2:1 | Amorphous | Excipient only |
| Hydroxypropyl | Methanol | 2:1 | Glass/oily | Glass/oily |
| Cellulose | | | substance | substance |
| Methyl | Water | 2:1 | Amorphous | N/A |
| Cellulose | | | | |
| Hydroxy Propyl | Water | 2:1 | Amorphous | N/A |
| Methyl | | | | |
| Cellulose | | | | |
| Sodium dodecyl | Methanol | 2:1 | Excipient only | Excipient only |
| Sulfate | | | | |
| L-Ascorbic Acid | Methanol | 2:1 | Glass/oily | Glass/oily |
| | | | substance | substance |
| Beta- | Water | 2:1 | Poor | N/A |
| Cyclodextrin | | | crystallinity | |
| (2- | Methanol | 2:1 | Amorphous | Glass/oily |
| hydroxypropyl) | | | | substance |
| Beta- | | | | |
| Cyclodextrin | | | | |
| Cellulose acetate | Methanol | 2:1 | Glass/oily | Glass/oily |
| | | | substance | substance |
| Cellulose acetate | Methanol | 2:1 | Glass/oily | Glass/oily |
| phthalate | | | substance | substance |
| PEG 1000 | Methanol | 2:1 | Glass/oily | Excipient only |
| | | | substance | |
| PEG 10000 | Water | 2:1 | Excipient only | N/A |
| Polyvinyl | Methanol | 2:1 | Glass/oily | Glass/oily |
| Pyrrolidone | | | substance | substance |
| (K85-K95) | | | | |
| Poloxamer 407 | Methanol | 2:1 | Glass/oily | Glass/oily |
| (Pluronic F127) | | | substance | substance |
| Butylated | Methanol | 2:1 | Glass/oily | Glass/oily |
| hydroxyanisole | | | substance | substance |
| Monostearine | Methanol | 2:1 | Excipient only | Poor crystallinity |

The solids labelled “Glass/oily substance” are amorphous, but were not tested for stability. The solids labelled “Amorphous” are stored under conditions of 40° C. and 75% relative humidity for 1 week, and stability is checked by XRPD at different time points. Results confirm that each of the solids labelled “amorphous” remained stable through 1 week under these accelerated aging conditions.

### Example 2: Free Base Containing Amorphous Solid Dispersions Created Using Evaporation

5 mg of the Compound 1 Free Base is dissolved in acetone (500 μl), dichloromethane (500 μl), or acetone/ethanol (3:1); and the excipients are dissolved in either water (500 μl) or methanol (500 μl), depending on their solubilities. The Free Base solution and excipient solution are added to each other. Some drops of water are added for the experiments that did not contain water. The samples are subsequently stored in vacuum (0 mbar, RT) for quick evaporation of the solvents. The experiments are performed using molar ratios of Free Base to excipient of 1:1, 1:2 and 2:1. Visual appearance is recorded and XRPD is measured for observed solids. The experiments marked with an “N/A” were not carried out due to immiscibility between dichloromethane and water.

TABLE 4
Evaporation experiments with Free Base:excipient ratio of 1:1

| | | Molar Ratio | Experimental Results | | |
|---|---|---|---|---|---|
| | Excipient | (Free | | | Acetone/EtOH |
| Excipient | Solvent | Base:Excipient) | Acetone | Dichloromethane | (3:1) |
| Cellulose | Methanol | 1:1 | Amorphous | Amorphous | Glass/oily |
| | | | | | substance |
| Hydroxypropyl | Methanol | 1:1 | Glass/oily | Glass/oily | Glass/oily |
| Cellulose | | | substance | substance | substance |
| Methyl | Water | 1:1 | Amorphous | N/A | Amorphous |
| Cellulose | | | | | |
| Hydroxy | Water | 1:1 | Amorphous | N/A | Glass/oily |
| Propyl Methyl | | | | | substance |
| Cellulose | | | | | |
| Sodium | Methanol | 1:1 | Crystal | Excipient only | Excipient only |
| dodecyl Sulfate | | | | | |
| L-Ascorbic | Methanol | 1:1 | Amorphous | Crystal | Glass/oily |
| Acid | | | | | substance |
| Beta- | Water | 1:1 | Poor | N/A | Amorphous |
| Cyclodextrin | | | crystallinity | | |
| (2- | Methanol | 1:1 | Amorphous | Amorphous | Amorphous |
| hydroxypropyl) | | | | | |
| Beta- | | | | | |
| Cyclodextrin | | | | | |
| Cellulose | Methanol | 1:1 | Glass/oily | Glass/oily | Glass/oily |
| acetate | | | substance | substance | substance |
| Cellulose | Methanol | 1:1 | Glass/oily | Glass/oily | Glass/oily |
| acetate | | | substance | substance | substance |
| phthalate | | | | | |
| PEG 1000 | Methanol | 1:1 | Excipient | Glass/oily | Excipient only |
| | | | only | substance | |
| PEG 10000 | Water | 1:1 | Excipient | N/A | Excipient only |
| | | | only | | |
| Polyvinyl | Methanol | 1:1 | Glass/oily | Glass/oily | Glass/oily |
| Pyrrolidone | | | substance | substance | substance |
| (K85-K95) | | | | | |
| Poloxamer 407 | Methanol | 1:1 | Glass/oily | Glass/oily | Glass/oily |
| (Pluronic | | | substance | substance | substance |
| F127) | | | | | |
| Butylated | Methanol | 1:1 | Glass/oily | Glass/oily | Glass/oily |
| hydroxyanisole | | | substance | substance | substance |
| Monostearine | Methanol | 1:1 | Amorphous | Poor crystallinity | Poor |
| | | | | | crystallinity |

TABLE 5
Evaporation experiments with Free Base:excipient ratio of 1:2

| | | Molar Ratio | Experimental Results | | |
|---|---|---|---|---|---|
| | Excipient | (Free | | | Acetone/EtOH |
| Excipient | Solvent | Base:Excipient) | Acetone | Dichloromethane | (3:1) |
| Cellulose | Methanol | 1:2 | Excipient | Excipient only | Excipient only |
| | | | only | | |
| Hydroxypropyl | Methanol | 1:2 | Glass/oily | Glass/oily | Glass/oily |
| Cellulose | | | substance | substance | substance |
| Methyl | Water | 1:2 | Amorphous | N/A | Amorphous |
| Cellulose | | | | | |
| Hydroxy | Water | 1:2 | Glass/oily | N/A | Amorphous |
| Propyl Methyl | | | substance | | |
| Cellulose | | | | | |
| Sodium | Methanol | 1:2 | Excipient | Excipient only | Excipient only |
| dodecyl Sulfate | | | only | | |
| L-Ascorbic | Methanol | 1:2 | Glass/oily | Glass/oily | Glass/oily |
| Acid | | | substance | substance | substance |
| Beta- | Water | 1:2 | Excipient | N/A | Excipient only |
| Cyclodextrin | | | only | | |
| (2- | Methanol | 1:2 | Amorphous | Amorphous | Amorphous |
| hydroxypropyl) | | | | | |
| Beta- | | | | | |
| Cyclodextrin | | | | | |
| Cellulose | Methanol | 1:2 | Glass/oily | Glass/oily | Glass/oily |
| acetate | | | substance | substance | substance |
| Cellulose | Methanol | 1:2 | Glass/oily | Glass/oily | Glass/oily |
| acetate | | | substance | substance | substance |
| phthalate | | | | | |
| PEG 1000 | Methanol | 1:2 | Excipient | Excipient only | Excipient only |
| | | | only | | |
| PEG 10000 | Water | 1:2 | Excipient | N/A | Excipient only |
| | | | only | | |
| Polyvinyl | Methanol | 1:2 | Glass/oily | Glass/oily | Glass/oily |
| Pyrrolidone | | | substance | substance | substance |
| (K85-K95) | | | | | |
| Poloxamer 407 | Methanol | 1:2 | Glass/oily | Glass/oily | Excipient only |
| (Pluronic | | | substance | substance | |
| F127) | | | | | |
| Butylated | Methanol | 1:2 | Glass/oily | Glass/oily | Glass/oily |
| hydroxyanisole | | | substance | substance | substance |
| Monostearine | Methanol | 1:2 | Excipient | Excipient only | Excipient only |
| | | | only | | |

TABLE 6
Evaporation experiments with Free Base:excipient ratio of 2:1

| | | Molar Ratio | Experimental Results | | |
|---|---|---|---|---|---|
| | Excipient | (Free | | | Acetone/EtOH |
| Excipient | Solvent | Base:Excipient) | Acetone | Dichloromethane | (3:1) |
| Cellulose | Methanol | 2:1 | Excipient | Amorphous | Amorphous |
| | | | only | | |
| Hydroxypropyl | Methanol | 2:1 | Glass/oily | Glass/oily | Glass/oily |
| Cellulose | | | substance | substance | substance |
| Methyl | Water | 2:1 | Amorphous | N/A | Amorphous |
| Cellulose | | | | | |
| Hydroxy | Water | 2:1 | Amorphous | N/A | Glass/oily |
| Propyl Methyl | | | | | substance |
| Cellulose | | | | | |
| Sodium | Methanol | 2:1 | Excipient | Excipient only | Poor |
| dodecyl Sulfate | | | only | | crystallinity |
| L-Ascorbic | Methanol | 2:1 | Glass/oily | Glass/oily | Amorphous |
| Acid | | | substance | substance | |
| Beta- | Water | 2:1 | Crystal | N/A | Crystal form |
| Cyclodextrin | | | form | | |
| (2- | Methanol | 2:1 | Amorphous | Amorphous | Amorphous |
| hydroxypropyl) | | | | | |
| Beta- | | | | | |
| Cyclodextrin | | | | | |
| Cellulose | Methanol | 2:1 | Amorphous | Glass/oily | Excipient only |
| acetate | | | | substance | |
| Cellulose | Methanol | 2:1 | Glass/oily | Glass/oily | Amorphous |
| acetate | | | substance | substance | |
| phthalate | | | | | |
| PEG 1000 | Methanol | 2:1 | Excipient | Excipient only | Excipient only |
| | | | only | | |
| PEG 10000 | Water | 2:1 | Excipient | N/A | Excipient only |
| | | | only | | |
| Polyvinyl | Methanol | 2:1 | Glass/oily | Glass/oily | Glass/oily |
| Pyrrolidone | | | substance | substance | substance |
| (K85-K95) | | | | | |
| Poloxamer 407 | Methanol | 2:1 | Glass/oily | Glass/oily | Excipient only |
| (Pluronic | | | substance | substance | |
| F127) | | | | | |
| Butylated | Methanol | 2:1 | Glass/oily | Glass/oily | Glass/oily |
| hydroxyanisole | | | substance | substance | substance |
| Monostearine | Methanol | 2:1 | Amorphous | Crystal form | Free base only |

The solids labelled “Glass/oily substance” are amorphous, but were not tested for stability. The solids labelled “Amorphous” are stored under conditions of 40° C. and 75% relative humidity for 1 week, and stability is checked by XRPD at different time points. Several amorphous patterns are observed, depending on solvent and ratio. The stability experiments show conversion into a crystalline pattern for some of the experiments. The products that remained amorphous are listed below.

TABLE 7
Evaporation experiments resulting in a stable amorphous
solid dispersion of Free Base

| | Molar Ratio (Free | |
|---|---|---|
| Excipient | base:Excipient) | Solvent |
| Cellulose | 1:1 | Acetone |
| Methyl Cellulose | 1:1 | Acetone |
| | 1:2 | Acetone/EtOH |
| | 2:1 | Acetone |
| | | Acetone/EtOH |
| Hydroxypropyl methyl | 1:1 | Acetone |
| cellulose | 1:2 | Acetone/EtOH |
| | 2:1 | Acetone |
| | | Acetone/EtOH |
| (2-hydroxypropyl) beta- | 1:1 | Acetone |
| cyclodextrin | | Acetone/EtOH |
| | | DCM |
| | 1:2 | Acetone |
| | 2:1 | DCM |
| L-ascorbic acid | 1:1 | Acetone |

### Example 3: Phosphate Salt Containing Amorphous Solid Dispersions Created Using Lyophilization

5 mg of (the mono-phosphate salt of Compound 1 is dissolved in acetonitrile/water (9:1)(500 μl) and the excipients are dissolved in either water (500 μl) or methanol (500 μl), depending on their solubilities. The phosphate solution and excipient solution are added to each other. Some drops of water are added for the experiments that did not contain water. The samples are subsequently stored in a freezer (−20° C.) for 16 hours and thereafter stored in vacuum (0 mbar, RT) for quick evaporation of the solvents. The experiments are performed using molar ratios of phosphate to excipient of 1:1, 1:2 and 2:1. Visual appearance is recorded and XRPD is measured for observed solids.

TABLE 8
Lyophilization experiments with phosphate:excipient ratio of 1:1

| | | | Experimental |
|---|---|---|---|
| | | Molar Ratio | Results - |
| Excipient | Excipient Solvent | (Phosphate:Excipient) | ACN/Water (9:1) |
| Cellulose | Methanol | 1:1 | Amorphous |
| Hydroxypropyl | Methanol | 1:1 | Glass/oily substance |
| Cellulose | | | |
| Methyl Cellulose | Methanol | 1:1 | Amorphous |
| Hydroxy Propyl | Water | 1:1 | Amorphous |
| Methyl Cellulose | | | |
| Sodium dodecyl | Methanol | 1:1 | Poor crystallinity |
| Sulfate | | | |
| L-Ascorbic Acid | Methanol | 1:1 | Glass/oily substance |
| Beta-Cyclodextrin | Water | 1:1 | Excipient only |
| (2-hydroxypropyl) | Methanol | 1:1 | Glass/oily substance |
| Beta-Cyclodextrin | | | |
| Cellulose acetate | Methanol | 1:1 | Glass/oily substance |
| Cellulose acetate | Methanol | 1:1 | Glass/oily substance |
| phthalate | | | |
| PEG 1000 | Methanol | 1:1 | Excipient only |
| PEG 10000 | Water | 1:1 | Excipient only |
| Polyvinyl Pyrrolidone | Methanol | 1:1 | Glass/oily substance |
| (K85-K95) | | | |
| Poloxamer 407 | Methanol | 1:1 | Crystal form |
| (Pluronic F127) | | | |
| Butylated | Methanol | 1:1 | Glass/oily substance |
| hydroxyanisole | | | |
| Monostearine | Methanol | 1:1 | Crystal form |

TABLE 9
Lyophilization experiments with phosphate:excipient ratio of 1:2

| | | | Experimental |
|---|---|---|---|
| | | Molar Ratio | Results - |
| Excipient | Excipient Solvent | (Phosphate:Excipient) | ACN/Water (9:1) |
| Cellulose | Methanol | 1:2 | Excipient only |
| Hydroxypropyl | Methanol | 1:2 | Glass/oily substance |
| Cellulose | | | |
| Methyl Cellulose | Methanol | 1:2 | Amorphous |
| Hydroxy Propyl | Water | 1:2 | Amorphous |
| Methyl Cellulose | | | |
| Sodium dodecyl | Methanol | 1:2 | Excipient only |
| Sulfate | | | |
| L-Ascorbic Acid | Methanol | 1:2 | Glass/oily substance |
| Beta-Cyclodextrin | Water | 1:2 | Excipient only |
| (2-hydroxypropyl) | Methanol | 1:2 | Glass/oily substance |
| Beta-Cyclodextrin | | | |
| Cellulose acetate | Methanol | 1:2 | Amorphous |
| Cellulose acetate | Methanol | 1:2 | Glass/oily substance |
| phthalate | | | |
| PEG 1000 | Methanol | 1:2 | Excipient only |
| PEG 10000 | Water | 1:2 | Excipient only |
| Polyvinyl Pyrrolidone | Methanol | 1:2 | Glass/oily substance |
| (K85-K95) | | | |
| Poloxamer 407 | Methanol | 1:2 | Excipient only |
| (Pluronic F127) | | | |
| Butylated | Methanol | 1:2 | Glass/oily substance |
| hydroxyanisole | | | |
| Monostearine | Methanol | 1:2 | Crystal form |

TABLE 10
Lyophilization experiments with phosphate:excipient ratio of 2:1

| | | | Experimental |
|---|---|---|---|
| | | Molar Ratio | Results - |
| Excipient | Excipient Solvent | (Phosphate:Excipient) | ACN/Water (9:1) |
| Cellulose | Methanol | 2:1 | Phosphate only |
| Hydroxypropyl | Methanol | 2:1 | Phosphate only |
| Cellulose | | | |
| Methyl Cellulose | Methanol | 2:1 | Amorphous |
| Hydroxy Propyl | Water | 2:1 | Amorphous |
| Methyl Cellulose | | | |
| Sodium dodecyl | Methanol | 2:1 | Phosphate only |
| Sulfate | | | |
| L-Ascorbic Acid | Methanol | 2:1 | Phosphate only |
| Beta-Cyclodextrin | Water | 2:1 | Poor crystallinity |
| (2-hydroxypropyl) | Methanol | 2:1 | Phosphate only |
| Beta-Cyclodextrin | | | |
| Cellulose acetate | Methanol | 2:1 | Glass/oily substance |
| Cellulose acetate | Methanol | 2:1 | Glass/oily substance |
| phthalate | | | |
| PEG 1000 | Methanol | 2:1 | Glass/oily substance |
| PEG 10000 | Water | 2:1 | Excipient only |
| Polyvinyl Pyrrolidone | Methanol | 2:1 | Glass/oily substance |
| (K85-K95) | | | |
| Poloxamer 407 | Methanol | 2:1 | Excipient only |
| (Pluronic F127) | | | |
| Butylated | Methanol | 2:1 | Glass/oily substance |
| hydroxyanisole | | | |
| Monostearine | Methanol | 2:1 | Phosphate only |

The solids labelled “Glass/oily substance” are amorphous, but were not tested for stability. The solids labelled “Amorphous” are stored under conditions of 40° C. and 75% relative humidity for 1 week, and stability is checked by XRPD at different time points. Results confirm that each of the solids labelled “amorphous” remained stable through 1 week under these accelerated aging conditions.

### Example 4: Phosphate Salt Containing Amorphous Solid Dispersions Created Using Evaporation

5 mg of the Compound 1 phosphate is dissolved in acetonitrile/water 9:1 (500 μl), tetrahydrofuran/water 9:1 (500 μl), or methanol/water 9:1 (500 μl) and the excipients are dissolved in either water or methanol (500 μl), depending on their solubilities. The phosphate solution and excipient solution are added to each other. The samples are subsequently stored in vacuum (0 mbar, RT) for quick evaporation of the solvents. The experiments are performed using molar ratios of phosphate to excipient of 1:1, 1:2 and 2:1. Visual appearance is recorded and XRPD is measured for observed solids.

TABLE 11
Evaporation experiments with phosphate:excipient ratio of 1:1

| | | Molar | Experimental Results | | |
|---|---|---|---|---|---|
| | Excipient | Ratio (Free | ACN/Water | THF/Water | MeOH:Water |
| Excipient | Solvent | Base:Excipient) | (9:1) | (9:1) | (9:1) |
| Cellulose | Methanol | 1:1 | Amorphous | Amorphous | Amorphous |
| Hydroxypropyl | Methanol | 1:1 | Amorphous | Amorphous | Amorphous |
| Cellulose | | | | | |
| Methyl | Water | 1:1 | Amorphous | Amorphous | Amorphous |
| Cellulose | | | | | |
| Hydroxy | Water | 1:1 | Amorphous | Amorphous | Amorphous |
| Propyl Methyl | | | | | |
| Cellulose | | | | | |
| Sodium | Methanol | 1:1 | Poor | Poor | Glass/oily |
| dodecyl Sulfate | | | crystallinity | crystallinity | substance |
| L-Ascorbic | Methanol | 1:1 | Glass/oily | Glass/oily | Glass/oily |
| Acid | | | substance | substance | substance |
| Beta- | Water | 1:1 | Poor | Poor | Poor |
| Cyclodextrin | | | crystallinity | crystallinity | crystallinity |
| (2- | Methanol | 1:1 | Glass/oily | Amorphous | Glass/oily |
| hydroxypropyl) | | | substance | | substance |
| Beta- | | | | | |
| Cyclodextrin | | | | | |
| Cellulose | Methanol | 1:1 | Amorphous | Glass/oily | Amorphous |
| acetate | | | | substance | |
| Cellulose | Methanol | 1:1 | Amorphous | Amorphous | Amorphous |
| acetate | | | | | |
| phthalate | | | | | |
| PEG 1000 | Methanol | 1:1 | Excipient | Excipient only | Excipient only |
| | | | only | | |
| PEG 10000 | Water | 1:1 | Excipient | Excipient only | Excipient only |
| | | | only | | |
| Polyvinyl | Methanol | 1:1 | Glass/oily | Amorphous | Glass/oily |
| Pyrrolidone | | | substance | | substance |
| (K85-K95) | | | | | |
| Poloxamer 407 | Methanol | 1:1 | Phosphate | Crystal form | Crystal form |
| (Pluronic | | | only | | |
| F127) | | | | | |
| Butylated | Methanol | 1:1 | Glass/oily | Glass/oily | Glass/oily |
| hydroxyanisole | | | substance | substance | substance |
| Monostearine | Methanol | 1:1 | Poor | Poor | Glass/oily |
| | | | crystallinity | crystallinity | substance |

TABLE 12
Evaporation experiments with phosphate:excipient ratio of 1:2

| | | Molar Ratio | Experimental Results | | |
|---|---|---|---|---|---|
| | Excipient | (Free | ACN/Water | THF/Water | MeOH:Water |
| Excipient | Solvent | Base:Excipient) | (9:1) | (9:1) | (9:1) |
| Cellulose | Methanol | 1:2 | Excipient | Excipient only | Poor |
| | | | only | | crystallinity |
| Hydroxypropyl | Methanol | 1:2 | Glass/Oily | Glass/Oily | Glass/Oily |
| Cellulose | | | substance | substance | substance |
| Methyl | Water | 1:2 | Amorphous | Amorphous | Amorphous |
| Cellulose | | | | | |
| Hydroxy | Water | 1:2 | Amorphous | Amorphous | Amorphous |
| Propyl Methyl | | | | | |
| Cellulose | | | | | |
| Sodium | Methanol | 1:2 | Excipient | Excipient only | Excipient only |
| dodecyl Sulfate | | | only | | |
| L-Ascorbic | Methanol | 1:2 | Glass/Oily | Glass/Oily | Glass/Oily |
| Acid | | | substance | substance | substance |
| Beta- | Water | 1:2 | Excipient | Glass/Oily | Crystal form |
| Cyclodextrin | | | only | substance | |
| (2- | Methanol | 1:2 | Glass/Oily | Amorphous | Glass/Oily |
| hydroxypropyl) | | | substance | | substance |
| Beta- | | | | | |
| Cyclodextrin | | | | | |
| Cellulose | Methanol | 1:2 | Amorphous | Amorphous | Crystal form |
| acetate | | | | | |
| Cellulose | Methanol | 1:2 | Glass/Oily | Glass/Oily | Glass/Oily |
| acetate | | | substance | substance | substance |
| phthalate | | | | | |
| PEG 1000 | Methanol | 1:2 | Crystal form | Glass/Oily | Excipient only |
| | | | | substance | |
| PEG 10000 | Water | 1:2 | Excipient | Excipient only | Excipient only |
| | | | only | | |
| Polyvinyl | Methanol | 1:2 | Glass/Oily | Glass/Oily | Glass/Oily |
| Pyrrolidone | | | substance | substance | substance |
| (K85-K95) | | | | | |
| Poloxamer 407 | Methanol | 1:2 | Excipient | Excipient only | Excipient only |
| (Pluronic | | | only | | |
| F127) | | | | | |
| Butylated | Methanol | 1:2 | Glass/Oily | Glass/Oily | Glass/Oily |
| hydroxyanisole | | | substance | substance | substance |
| Monostearine | Methanol | 1:2 | Glass/Oily | Crystal form | Glass/Oily |
| | | | substance | | substance |

TABLE 13
Evaporation experiments with phosphate:excipient ratio of 2:1

| | | Molar Ratio | Experimental Results | | |
|---|---|---|---|---|---|
| | Excipient | (Free | ACN/Water | THF/Water | MeOH:Water |
| Excipient | Solvent | Base:Excipient) | (9:1) | (9:1) | (9:1) |
| Cellulose | Methanol | 2:1 | Amorphous | Amorphous | Poor |
| | | | | | crystallinity |
| Hydroxypropyl | Methanol | 2:1 | Glass/oily | Amorphous | Glass/oily |
| Cellulose | | | substance | | substance |
| Methyl | Water | 2:1 | Amorphous | Amorphous | Amorphous |
| Cellulose | | | | | |
| Hydroxy | Water | 2:1 | Glass/oily | Amorphous | Amorphous |
| Propyl Methyl | | | substance | | |
| Cellulose | | | | | |
| Sodium | Methanol | 2:1 | Crystal form | Crystal form | Amorphous |
| dodecyl Sulfate | | | | | |
| L-Ascorbic | Methanol | 2:1 | Amorphous | Amorphous | Glass/oily |
| Acid | | | | | substance |
| Beta- | Water | 2:1 | Crystal form | Crystal form | Crystal form |
| Cyclodextrin | | | | | |
| (2- | Methanol | 2:1 | Amorphous | Amorphous | Amorphous |
| hydroxypropyl) | | | | | |
| Beta- | | | | | |
| Cyclodextrin | | | | | |
| Cellulose | Methanol | 2:1 | Amorphous | Amorphous | Glass/oily |
| acetate | | | | | substance |
| Cellulose | Methanol | 2:1 | Amorphous | Glass/oily | Amorphous |
| acetate | | | | substance | |
| phthalate | | | | | |
| PEG 1000 | Methanol | 2:1 | Crystal form | Glass/oily | Crystal form |
| | | | | substance | |
| PEG 10000 | Water | 2:1 | Excipient | Excipient only | Excipient only |
| | | | only | | |
| Polyvinyl | Methanol | 2:1 | Amorphous | Amorphous | Poor |
| Pyrrolidone | | | | | crystallinity |
| (K85-K95) | | | | | |
| Poloxamer 407 | Methanol | 2:1 | Excipient | Excipient only | Excipient only |
| (Pluronic | | | only | | |
| F127) | | | | | |
| Butylated | Methanol | 2:1 | Glass/oily | Glass/oily | Glass/oily |
| hydroxyanisole | | | substance | substance | substance |
| Monostearine | Methanol | 2:1 | Phosphate | Phosphate | Phosphate only |
| | | | only | only | |

The solids labelled “Glass/oily substance” are amorphous, but were not tested for stability. The solids labelled “Amorphous” are stored under conditions of 40° C. and 75% relative humidity for 1 week, and stability is checked by XRPD at different time points. Several amorphous patterns are observed, depending on solvent and ratio. The stability experiments show conversion into a crystalline pattern for some of the experiments. The products that remained amorphous are listed below.

| | Molar Ratio | |
|---|---|---|
| Excipient | (Free base:Excipient) | Solvent |
| Cellulose | 1:1 | THF/Water |
| | | MeOH/Water |
| | 2:1 | ACN/Water |
| | | THF/Water |
| Hydroxypropyl cellulose | 1:1 | ACN/Water |
| | | THF/Water |
| | | MeOH/Water |
| | 2:1 | THF/Water |
| Methyl Cellulose | 1:1 | ACN/Water |
| | | THF/Water |
| | | MeOH/Water |
| | 1:2 | ACN/Water |
| | | THF/Water |
| | | MeOH/Water |
| | 2:1 | ACN/Water |
| | | THF/Water |
| | | MeOH/Water |
| Hydroxypropyl methyl | 1:1 | ACN/Water |
| cellulose | | THF/Water |
| | | MeOH/Water |
| | 1:2 | ACN/Water |
| | | THF/Water |
| | 2:1 | THF/Water |
| (2-hydroxypropyl) beta- | 1:1 | THF/Water |
| cyclodextrin | 1:2 | THF/Water |
| | 2:1 | MeOH/Water |
| Cellulose acetate | 1:1 | ACN/Water |
| | | MeOH/Water |
| | 1:2 | THF/Water |
| | 2:1 | ACN/Water |
| | | THF/Water |
| Cellulose acetate phthalate | 1:1 | ACN/Water |
| | | THF/Water |
| | | MeOH/Water |
| | 2:1 | MeOH/Water |
| Polyvinyl pyrrolidone | 2:1 | ACN/Water |
| | | THF/Water |

1. An amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free base or pharmaceutically acceptable salt form and an excipient.

2. The amorphous solid dispersion according to claim 1, wherein Compound 1 is in free base form.

3. The amorphous solid dispersion according to claim 1, wherein Compound 1 is in phosphate salt form.

4. The amorphous solid dispersion according to claim 1, wherein the excipient comprises a cellulose; polyethylene glycol (e.g., polyethylene glycol having a molecular weight of 500-50000 Daltons, e.g., PEG 1000 or PEG 10000); monostearine; polyvinyl pyrrolidone; PEG/PPG block co-polymers (e.g., a poloxamer (e.g., poloxamer 407, pluronic F127)); ascorbic acid (i.e., L-ascorbic acid); butylated hydroxyanisole; sodium dodecyl sulfate; a cyclodextrin (e.g., beta cyclodextrin, 2-Hydroxy propyl)-β-cyclodextrin); or combinations thereof.

5. The amorphous solid dispersion according to claim 1, wherein the excipient comprises cellulose; hydroxypropyl cellulose; methyl cellulose; hydroxy propyl methyl cellulose; sodium dodecyl sulfate; ascorbic acid (e.g., L-ascorbic acid); beta-cyclodextrin; (2-hydroxypropyl) beta-cyclodextrin; cellulose acetate; cellulose acetate phthalate; polyethylene glycol (e.g., polyethylene glycol having a molecular weight of 500-50000 Daltons, e.g., PEG 1000 or PEG 10000); PEG/PPG block co-polymers (e.g., a poloxamer (e.g., poloxamer 407, pluronic F127)); butylated hydroxyanisole; monostearine; or combinations thereof.

6. The amorphous solid dispersion according to claim 1, wherein the excipient comprises cellulose, methyl cellulose; hydroxy propyl methyl cellulose; (2-hydroxypropyl) beta-cyclodextrin; cellulose acetate; cellulose acetate phthalate; polyvinyl pyrrolidone (e.g., K85-K95); ascorbic acid (e.g., L-ascorbic acid); or combinations thereof.

7. The amorphous solid dispersion according to claim 1, wherein Compound 1 and the excipient are present in a molar ratio between 1:5 to 5:1, e.g., a molar ratio between 1:2 and 2:1, e.g., 1:1, 1:2, or 2:1.

8. The amorphous solid dispersion according to claim 1, wherein the amorphous solid dispersion is in dosage form, which contains Compound 1 in an amount of about 0.5 mg to about 300 mg, wherein the amount is calculated as the free base equivalent.

9. The amorphous solid dispersion according to claim 1, wherein the amorphous solid dispersion is in dosage form, which contains Compound 1 in an amount of about 30 mg to about 90 mg, wherein the amount is calculated as the free base equivalent.

10. The amorphous solid dispersion according to claim 1, wherein the amorphous solid dispersion is stable for a period of about one day, about three days, or about 7 days under accelerated aging conditions (e.g., 40° C. and 75% relative humidity).

11. The amorphous solid dispersion according to claim 1, wherein Compound 1 is amorphous and contains less than 10 wt. % crystalline forms of Compound 1, e.g., less than 5 wt. % crystalline forms of Compound 1, e.g., less than 1 wt. % crystalline forms of Compound 1, e.g., less than 0.1 wt. % crystalline forms of Compound 1, e.g., less than 0.01 wt. % crystalline forms of compound 1, e.g., essentially free of crystalline forms of Compound 1.

12. A method of making an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) and an excipient according to claim 1, the method comprising the steps of:
a) dissolving Compound 1 in a first solvent;
b) dissolving the excipient in a second solvent;
c) combining the solutions from steps a) and b); and
d) removing the solvents from the mixture.

13. The method according to claim 12, wherein the first solvent comprises one or more of water, acetone, dichloromethane, an alcohol (e.g., methanol or ethanol), dioxane, tetrahydrofuran, acetonitrile, and combinations thereof.

14. The method according to claim 12, wherein the first solvent is acetone; dichloromethane; acetone and ethanol (e.g., in a 3:1 ratio); tetrahydrofuran and water (e.g., in a 9:1 ratio); methanol and water (e.g., in a ratio of 9:1); or acetonitrile and water (e.g., in a ratio of 9:1).

15. The method according to claim 12, wherein the second solvent is water and/or methanol.

16. The method according to claim 12, wherein removing the solvents from the mixture comprises lyophilization and/or evaporation.

17. The method according to claim 12, wherein the removal step comprises lyophilization and evaporation.

18. The method according to claim 12, wherein the removal step comprises freezing the mixture at a temperature at or below 0° C., e.g., at or below −10° C., e.g., at or below −20° C., followed by evaporation of the solvent at reduced pressure (e.g., 0 bar).

19. The method according to claim 12, wherein the removal step comprises evaporation of the solvent at reduced pressure (e.g., 0 bar).

20. A method for the prophylaxis or treatment of a patient, e.g., a human, suffering from a disorder selected from the following:
i. Neurodegenerative diseases, including Parkinson's disease, restless leg, tremors, dyskinesias, Huntington's disease, Alzheimer's disease, and drug-induced movement disorders;
ii. Mental disorders, including depression, attention deficit disorder, attention deficit hyperactivity disorder, bipolar illness, anxiety, sleep disorders, e.g., narcolepsy, cognitive impairment, e.g., cognitive impairment of schizophrenia, dementia, Tourette's syndrome, autism, fragile X syndrome, psychostimulant withdrawal, and drug addiction;
iii. Circulatory and cardiovascular disorders, including cerebrovascular disease, stroke, congestive heart disease, hypertension, pulmonary hypertension, e.g., pulmonary arterial hypertension, and sexual dysfunction, including cardiovascular diseases and related disorders;
iv. Respiratory and inflammatory disorders, including asthma, chronic obstructive pulmonary disease, and allergic rhinitis, as well as autoimmune and inflammatory diseases;
v. Diseases that may be alleviated by the enhancement of progesterone-signaling such as female sexual dysfunction;
vi. A disease or disorder such as psychosis, glaucoma, or elevated intraocular pressure;
vii. Traumatic brain injury;
viii. Cancers or tumors, e.g., brain tumors, a glioma (e.g., ependymoma, astrocytoma, oligodendrogliomas, brain stem glioma, optic nerve glioma, or mixed gliomas, e.g., oligoastrocytomas), an astrocytoma (e.g., glioblastoma multiforme), osteosarcoma, melanoma, leukemia, neuroblastoma or leukemia;
ix. Renal disorders, e.g., kidney fibrosis, chronic kidney disease, renal failure, glomerulosclerosis and nephritis;
x. Any disease or condition characterized by low levels of cAMP and/or cGMP (or inhibition of cAMP and/or cGMP signaling pathways) in cells expressing PDE1; and/or
xi. Any disease or condition characterized by reduced dopamine D1 receptor signaling activity,
the method comprising administering to a patient in need thereof a therapeutically effective amount of an amorphous solid dispersion comprising (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one (Compound 1) in free base or pharmaceutically acceptable salt form and an excipient according to claim 1.
